(19) United States
(12) Patent Application Publication
(10) Pub. No.: US 2011/0046426 A1
(43) Pub. Date: Feb. 24, 2011

(54) METHOD FOR CONTINUOUS CONVERSION OF METHANOL TO HIGHER HYDROCARBONS AND CATALYST USED THEREIN

(75) Inventor: William PORTERFIELD, Hampden-Sydney, VA (US)

Correspondence Address: MCGUIREWOODS, LLP, 1750 TYSONS BLVD, SUITE 1800, MCLEAN, VA, 22102, US

(73) Assignee: Hampden-Sydney College, Hampden-Sydney, VA (US)

(21) Appl. No.: 12/858,648

(22) Filed: Aug. 18, 2010

Related U.S. Application Data
(60) Provisional application No. 61/234,842, filed on Aug. 18, 2009.

(51) Int. Cl. C07C 1/20; B01J 19/00

(52) U.S. Cl. 585/469; 422/187

(57) ABSTRACT

Methods and apparatuses for converting methanol to higher hydrocarbons in a continuous process. A distillation column may be packed with inert material and filled with an ionic liquid. The ionic liquid may function as both reaction medium and catalyst. Derivative of zinc iodide and indium iodide may serve as the possible catalytic species. Higher hydrocarbons may be isolated from reaction effluent by condensation in a cold-water condenser, a cold trap, or both.

### CROSS-REFERENCE TO RELATED APPLICATIONS

This application claims priority under 35 U.S.C. §119(e) to provisional U.S. Patent Application No. 61/234,842, filed on Aug. 18, 2009, the disclosure of which is expressly incorporated by reference herein in its entirety.

### BACKGROUND

1. Field of the Present Disclosure

The present disclosure is directed to a method, catalyst, and reaction medium for the continuous conversion of methanol to a mixture of higher hydrocarbons, from propene to hexamethylbenzene. Specifically, the method uses an ionic liquid as both the reaction medium and the catalyst.

2. Related Art

The industrialized world relies on hydrocarbons, also known as fossil fuels. Coal is burned to provide electricity, and crude oil is refined to produce gasoline, jet fuel, plastics, and so on. Supplies of fossil fuels are limited, and many experts predict that the world's known oil supplies will be depleted before the end of the 21st century. Thus, there is a critical need to develop alternative energy sources, including renewable energy.

One possibility is the conversion of methanol into a mixture of higher hydrocarbons, which can replace crude oil in the refining process. Methanol is normally produced from methane, the major component of natural gas. Renewable sources of methanol are also available, including methanol distillation from wood and biomethane production from, for example, algae biofuel or anaerobic fermentation of municipal waste.

In the past, the conversion reaction has been carried out by suspending the catalyst, solid zinc iodide, in liquid methanol, sealing the mixture in a pressure vessel, and heating to approximately 200° C. for approximately two hours. This is a batch process, which is slow and inefficient. Moreover, the batch process would be difficult and expensive to adapt to the high-volume industrial production necessary for viable alternative fuels.

Accordingly, there is a need for an inexpensive, continuous, and efficient process for converting methanol to higher hydrocarbons that can be adapted to large-scale production.

### SUMMARY

The present disclosure meets the foregoing need and allows conversion of methanol at atmospheric pressure using an ionic liquid as both solvent and catalyst, which results in a significant improvement in cost and efficiency and other advantages apparent from the discussion herein.

Accordingly, in one aspect of the present disclosure, a method for converting methanol to higher hydrocarbons is described. The method includes injecting methanol into a distillation column packed with inert material and filled with an ionic liquid, collecting the reaction effluent from the distillation column, and condensing the reaction effluent.

The ionic liquid may include an iodozincate ion. The ionic liquid may further include a cation selected from diethylpiperidinium and alkylated imidazolium ions. Additional zinc iodide may also be dissolved in the ionic liquid. Alternatively, the ionic may include an anion derived from zinc iodide or indium iodide. The reaction effluent may be condensed by passing the effluent to a cold-water condenser, a cold trap, or both. The cold trap may be cooled to a temperature of about −100° C. The distillation column may be heated to a temperature between about 175° C. and about 250° C., or the distillation column may be heated to a temperature between about 200° C. and 220° C.

According to another aspect of the present disclosure, an apparatus includes a distillation column configured to hold an ionic liquid that serves as both reaction medium and catalyst, an inert material arranged in the distillation column and surrounded by the ionic liquid, an injection device configured to receive methanol for injection into the distillation column, and a condenser configured to receive reaction effluent from the distillation column.

The ionic liquid may include an iodozincate ion and may include a cation selected from diethylpiperidinium and alkylated imidazolium ions. The condenser may include a cold-water condenser, a cold trap, or both. The cold trap may be cooled to about −100° C. The distillation column may be heated to a temperature between about 175° C. and about 250° C., or the distillation column may be heated to a temperature between about 200° C. and 220° C.

Additional features, advantages, and embodiments of the present disclosure may be set forth or apparent from consideration of the following detailed description, drawings, and claims. Moreover, it is to be understood that both the foregoing summary of the present disclosure and the following detailed description are exemplary and intended to provide further explanation without limiting the scope of the present disclosure as claimed.

### DETAILED DESCRIPTION

The embodiments of the present disclosure and the various features and advantageous details thereof are explained more fully with reference to the non-limiting embodiments and examples that are described and/or illustrated in the accompanying drawings and detailed in the following description. It should be noted that the features illustrated in the drawings are not necessarily drawn to scale, and features of one embodiment may be employed with other embodiments as the skilled artisan would recognize, even if not explicitly stated herein. Descriptions of well-known components and processing techniques may be omitted so as to not unnecessarily obscure the embodiments of the present disclosure. The examples used herein are intended merely to facilitate an understanding of ways in which the present disclosure may be practiced and to further enable those of skill in the art to practice the embodiments of the present disclosure. Accordingly, the examples and embodiments herein should not be construed as limiting the scope of the present disclosure, which is defined solely by the appended claims and applicable law. Moreover, it is noted that like reference numerals represent similar parts throughout the several views of the drawings.

According to an aspect of the present disclosure, an ionic liquid may serve as both the reaction medium and the reaction catalyst. Use of an ionic liquid as catalyst with a different reaction medium, as well as use of ionic liquid as reaction medium with a different catalyst, are contemplated and within the spirit and scope of the present disclosure. An exemplary liquid is diethylpiperidinium triiodozincate, (C₂H₅)₂C₅H₁₀N⁺ZnI₃^{—}. Additional zinc iodide may be dissolved in the solution in the range of about 50% to about 200% relative to the iodozincate ion. The catalytic species is either the iodozincate anion or dissolved zinc iodide, and any appropriate cation, as understood by one skilled in the art, may be substituted for the diethylpiperidinium in the above exemplary liquid. For example, alkylated imidzolium ions, including butylmethylimidazolium, may be used in the ionic liquid. Thus any ionic liquid iodozincate may be used to practice the present disclosure and is within the scope of the present disclosure.

The present disclosure may also be practiced by making an ionic liquid that includes indium analogs of the zinc compounds described above. According to an aspect of the present disclosure, the conversion reaction may use indium iodide InI₃ as the catalyst instead of zinc iodide.

According to another aspect of the present disclosure, the reaction may be performed in a distillation column. Other types of reaction vessels may also be used. The distillation column may be packed with inert material, such as, e.g., glass beads or glass rings. The column may be filled with the ionic liquid and heated by a heating device to a temperature between approximately 175° C. and 250° C. Preferably, the temperature is between approximately 200° C. and 220° C.

Methanol may be injected using an injection device near the base of the column, where it vaporizes and rises up through the column. The vaporization may be quick or immediate. The vaporized methanol may rise through the column in bubbles. Methanol may be injected continuously, or it may be injected incrementally or intermittently. The transit time for each bubble is preferably about 3 seconds. Transit time may be longer or shorter, depending on the size of the column, the viscosity of the ionic liquid, the density of the inert packing material, and/or other factors, without departing from the spirit and scope of the present disclosure. According to one aspect of the present disclosure, the transit time may be between about 1 second and about 10 seconds. According to an additional aspect of the present disclosure, the transit time may be as long as about 20 seconds or longer, as will be understood by one skilled in the art, depending on the particular requirements of a given application.

The gas exiting the top of the column, also known as reaction effluent, may contain a mixture of hydrocarbon and aqueous products. Using propene C₃H₆ as a typical product, a balanced reaction equation is

3CH₃OH→C₃H₆+3H₂O

Reaction effluent may be collected by a still head at the top of the column and passed to a cold-water condenser. The condenser may produce a two-phase condensate. One phase may be aqueous (water) while the other phase may be non-aqueous and may contain hydrocarbons. The effluent may alternatively or additionally be passed through a cold trap, which may be cooled to about −100° C. The cold trap may condense propene, butenes, and other components of effluent that are gases at room temperature.

### EXPERIMENTS

Three separate reactors were constructed. Each reactor consisted of a vertical Pyrex tube, about 1-2 cm diameter and at least 70 cm high, heated by a standard distillation-column heater. A small flask, roughly 10 or 15 mL in size, was fitted to the bottom of the reactor tube and a standard-taper still head was attached to the top. The small flask had a side-arm that allowed a silicone rubber septum to be mounted for hypodermic syringe injection of methanol. All joints were sealed using Teflon sleeves instead of grease, and both the flask and reactor tube were filled with glass packing (Raschig rings).

Methanol was injected at about 0.01 mL per injection. Injections of this volume could be made frequently whereas injections of 1 mL methanol required from 3 to 20 minutes, depending on the stage of the reaction. In addition to the liquid condensate received from a cold-water condenser on the still head, the reaction effluent was passed through a cold trap at about −100° C. The use of the cold trap allowed collection of propene and butenes, which are gases at room temperature.

Each reactor produced similar results. Liquid products were analyzed using gas-chromatography/mass-spectrometry (GC/MS) and the NIST MS database search software, followed by infrared (IR) and both ¹H and ¹³C nuclear magnetic resonance (NMR) characterization. GC/MS results indicate a variable number of products, between 5 and 25, for each reaction run. The reaction products include propene, methylated pentenes and hexenes, and heavier hydrocarbons up to pentamethylbenzene and hexamethylbenzene. Some unreacted methanol was also collected, as well as some iodomethane formed from methanol in the early stages of the reaction.

While the present disclosure has been described in terms of exemplary embodiments, those skilled in the art will recognize that the present disclosure can be practiced with modifications in the spirit and scope of the appended claims. These examples given above are merely illustrative and are not meant to be an exhaustive list of all possible designs, embodiments, applications or modifications of the present disclosure.

What is claimed is:

1. A method for converting methanol to higher hydrocarbons, the method comprising:
injecting methanol into a distillation column packed with inert material and filled with an ionic liquid;
collecting a reaction effluent from the distillation column; and
condensing the reaction effluent.

2. The method of claim 1, wherein the ionic liquid comprises an iodozincate anion.

3. The method of claim 2, wherein the ionic liquid comprises a cation selected from the group consisting of diethylpiperidinium and alkylated imidazolium ions.

4. The method of claim 2, further comprising dissolving additional zinc iodide in the ionic liquid.

5. The method of claim 1, wherein the ionic liquid comprises an anion derived from one of zinc iodide and indium iodide.

6. The method of claim 1, wherein condensing the reaction effluent comprises passing the effluent to a cold-water condenser.

7. The method of claim 6, further comprising passing the effluent to a cold trap.

8. The method of claim 7, wherein the cold trap is cooled to a temperature of about −100° C.

9. The method of claim 1, wherein condensing the reaction effluent comprises passing the effluent to a cold trap.

10. The method of claim 8, wherein the cold trap is cooled to a temperature of about −100° C.

11. The method of claim 1, further comprising heating the distillation column to a temperature between about 175° C. and about 250° C.

12. The method of claim 1, further comprising heating the distillation column to a temperature between about 200° C. and about 220° C.

13. An apparatus comprising:
a distillation column configured to hold an ionic liquid that serves as both reaction medium and catalyst;
an inert material arranged in the distillation column and surrounded by the ionic liquid;
an injection device configured to receive methanol for injection into the distillation column; and
a condenser configured to receive a reaction effluent from the distillation column.

14. The apparatus of claim 13, wherein the ionic liquid comprises an iodozincate anion.

15. The apparatus of claim 13, wherein the ionic liquid comprises a cation selected from the group consisting of diethylpiperidinium and alkylated imidazolium ions.

16. The apparatus of claim 13, wherein the condenser comprises a cold-water condenser.

17. The apparatus of claim 13, wherein the condenser comprises a cold trap.

18. The apparatus of claim 17, wherein the cold trap is cooled to a temperature of about −100° C.

19. The apparatus of claim 13, wherein the distillation column is heated to a temperature between about 175° C. and about 250° C.

20. The apparatus of claim 13, wherein the distillation column is heated to a temperature between about 200° C. and about 220° C.
